(19) United States
(12) Patent Application Publication
(10) Pub. No.: US 2012/0330313 A1
(43) Pub. Date: Dec. 27, 2012

(54) Intramedullary nail technology

(76) Inventors: John F. Grady, Burr Ridge, IL (US); Albert L. Lippincott, III, Prior Lake, MN (US); Bruce H. Ziran, Decatur, GA (US); Jay Evans, San Diego, CA (US)

(21) Appl. No.: 13/373,936

(22) Filed: Dec. 5, 2011

Related U.S. Application Data
(60) Provisional application No. 61/419,474, filed on Dec. 3, 2010.

(51) Int. Cl. A61B 17/58

(52) U.S. Cl. 606/64

(57) ABSTRACT

Embodiments of the invention provide intramedullary nail assemblies that provide particularly good internal fixation, are particularly easy to deploy, or both. Embodiments also provide methods of using such nail assemblies.

### CROSS-REFERENCE TO RELATED APPLICATIONS

This application claims the benefit of U.S. Provisional Application No. 61/419,474, filed Dec. 3, 2010, the content of which is hereby incorporated by reference in its entirety.

### FIELD

The present invention relates generally to bone nails such as intramedullary nails. Specifically, embodiments of this invention relates to intramedullary nails that provide enhanced internal fixation of one or more bones being treated, are particularly easy to deploy, or both.

### BACKGROUND

A variety of nail systems are known for treating fractured bones or fusing two or more bones. One type of nail system is an intramedullary nail system. Typically, a medullary canal is present in one or more long bones to be treated. The nail is then advanced axially into the canal, e.g., so as to extend beyond a fracture or joint space. In many cases, the nail has a plurality of transverse hole openings. Bone screws are passed through the bone and into these transverse hole openings in the nail. This anchors the nail to the bone and fixes the affected bone(s) in a desired position to facilitate fracture healing and/or fusing.

One problem with conventional intramedullary nail systems is that the screws and the rest of the nail system may not remain perfectly fixed to the nail and bone, thereby allowing undesired micromotion in the system. This, of course, can have negative effects on the healing or fusing process. It would be desirable to provide an intramedullary nail system that provides enhanced fixation to the bone(s) it is intended to stabilize.

Another problem with conventional nail systems is that the transverse screw holes in the nail tend to be limited in terms of their ability to properly receive the leading end of a screw advanced blindly through bone toward the inlet of such a screw hole. The transverse screw holes in many conventional nails are internally threaded all the way through the nail. As a result, when a screw is advanced blindly toward such a threaded hole, if the screw's orientation is slightly off its intended position, then the screw can veer off, missing its intended seat in the threaded hole. One existing nail system has screw holes with first and second regions where an insert is provided in the first region to receive and fixedly retain a screw while the second region has a conventional internal thread. Such systems, however, require that the screw hole be equipped with a separate insert with the added capability of locking the screw within the hole, and therefore are more complicated and expensive than a simple through-hole. It would be desirable to provide a screw hole that facilitates blind placement of a transverse screw.

### SUMMARY

Certain embodiments of the present invention provide a bone nail assembly comprising a bone nail having first and second ends (e.g., proximal and distal, or distal and proximal, respectively) and an elongated nail shaft extending between its ends. In the present embodiments, the nail shaft has at least one screw hole proximate the second end of the nail shaft and at least one screw hole proximate the first end of the nail shaft. Each of these screw holes passes through the nail shaft and is configured to receive a screw for fixing the nail within one or more desired bones. In certain embodiments, the nail shaft has a hollow internally threaded section adjacent its second end, and an anchoring sleeve surrounds this hollow internally threaded section of the nail shaft. In certain embodiments, the anchoring sleeve and the section of the nail shaft each have a transverse screw opening configured to receive a screw through both the anchoring sleeve and the section of the nail shaft that fixes axial movement between the anchoring sleeve and the nail shaft. The anchoring sleeve defines an internal cavity configured to receive a section of the nail shaft. In the present embodiments, the anchoring sleeve has at least one outwardly projecting fin, which is provided with a mount hole that is internally threaded so as to provide for positive attachment between the fin and a desired screw. This mount hole is configured to receive the desired screw for fixing the nail within the one or more bones to be treated. Preferably, the mount hole on the fin is spaced radially from a central axis of the nail shaft, thereby creating a moment arm between the desired screw and the central axis of the nail shaft. This provides resistance to micromotion of the nail assembly relative to the one or more bones being treated.

In some embodiments, the invention provides a bone nail assembly comprising a bone nail having proximal and distal ends and an elongated nail shaft extending between the ends. A screw hole passes through the nail shaft and is configured to receive a screw for fixing the nail within one or more desired bones. In certain embodiments, the screw hole comprises a threaded first portion and a non-threaded second portion. Preferably, the non-threaded second portion is bounded by a smooth wall defined by the nail shaft and being configured to receive a screw at different approach angles and guide such screw to the threaded first portion. The non-threaded second portion of the screw hole preferably is devoid of an insert occupying any portion of the screw hole.

Certain embodiments of the invention provide a bone nail assembly comprising a bone nail having proximal and distal ends and an elongated nail shaft extending between the ends. A screw hole passes through the nail shaft and is configured to receive a screw for fixing the nail within one or more desired bones. In certain embodiments, the screw hole comprises a substantially straight first portion and an angled second portion. Preferably, the angled second portion is bounded by a wall defined by the nail shaft that extends inward toward a center of the screw hole from the exterior surface of the nail shaft to the first portion of the screw hole. The wall is preferably configured to receive a screw at different approach angles and guide such screw to the threaded first portion. The angled second portion of the screw hole preferably is devoid of an insert occupying any portion of the screw hole.

Some preferred embodiments of the invention provide a bone nail assembly comprising a bone nail having proximal and distal ends and an elongated nail shaft extending between the ends. A screw hole passes through the nail shaft and is configured to receive a screw for fixing the nail within one or more desired bones. In the present embodiments, the screw hole comprises a threaded first portion and a non-threaded and/or angled second portion. Preferably, the second portion is bounded by a wall defined by the nail shaft and is exposed to receive a screw and guide the screw toward the threaded first portion. The second portion of the screw hole preferably is devoid of an insert occupying any portion of the screw hole. In certain embodiments, the nail shaft has a hollow internally threaded section adjacent its distal end, and an anchoring sleeve surrounds the hollow internally threaded section of the nail shaft. The anchoring sleeve defines an internal cavity configured to receive a section of the nail shaft. In the present embodiment, the anchoring sleeve has at least one outwardly projecting fin, which is provided with a mount hole internally threaded to provide for positive attachment between the fin and a desired screw. The mount hole is configured to receive the desired screw for fixing the nail within one or more bones to be treated. In the present embodiment, the mount hole on the fin is spaced radially from a central axis of the nail shaft, thereby creating a moment arm between the desired screw and the central axis of the nail shaft. This provides resistance to micromotion of the nail assembly relative to the one or more bones being treated.

These and various other features and advantages will be apparent from a reading of the following detailed description.

### BRIEF DESCRIPTION OF THE DRAWINGS

FIG. 1 is a first side view of an intramedullary nail system in accordance with certain embodiments of the present invention.

FIG. 2 is a cross-sectional view of the nail system of FIG. 1.

FIG. 3 is a second side view of the nail system of FIG. 1.

FIG. 4 is an exploded view of the nail system of FIG. 1.

FIG. 5 is a perspective detail view of a distal end region of the nail system of FIG. 1.

FIG. 6 is a first cross-sectional detail view of a distal end region of the nail shaft of FIG. 1.

FIG. 7 is a second cross-sectional detail view of the distal end region of the nail shaft of FIG. 1.

FIGS. 8a-8d are various detail views of a washer member of FIG. 1.

FIGS. 9a-9c are various detail views of an anchoring sleeve of FIG. 1.

FIG. 10 is a perspective view of the bone nail of FIG. 1.

FIGS. 11a and 11b are various detail views of an internal compression screw of FIGS. 2 and 4.

FIGS. 12a-12d are various detail views of an end cap in accordance with certain embodiments of the present invention.

FIG. 13 is a cross-sectional detail view of a distal end region of a nail shaft in accordance with certain embodiments of the present invention.

FIG. 14 is a cross-sectional detail view of a distal end region of a nail shaft in accordance with certain embodiments of the present invention.

FIG. 15 is a first side view of another intramedullary nail system in accordance with certain embodiments of the present invention.

FIG. 16 is an exploded view of the nail system of FIG. 15.

FIG. 17 is a perspective detail view of a distal end region of the nail system of FIG. 15.

FIG. 18 is another perspective view of the nail system of FIG. 15.

FIGS. 19a-19c are various detail views of the nail system of FIG. 15 in place for fixing a portion of a patient's skeletal foot anatomy.

FIG. 20 is a side view of another intramedullary nail system in accordance with certain embodiments of the present invention.

FIGS. 21a-21c are various detail views of the nail system of FIG. 20 in place within a proximal femoral neck.

FIG. 22 is an exploded view of another intramedullary nail system in accordance with certain embodiments of the present invention.

FIG. 23 is a side view of the nail system of FIG. 22.

FIGS. 24a-24c are various detail views of the nail system of FIG. 22 in place within a humerus proximal end.

FIGS. 25a-25c are various views of a nail system implanted within a femur in accordance with certain embodiments of the present invention.

FIGS. 26a-26c are various detail views of the nail system of FIGS. 25a-25c.

### DETAILED DESCRIPTION OF PREFERRED EMBODIMENTS

The following detailed description is to be read with reference to the drawings, in which like elements in different drawings have like reference numerals. The drawings, which are not necessarily to scale, depict selected embodiments and are not intended to limit the scope of the invention. Skilled artisans will recognize that the given examples have many useful alternatives, which fall within the scope of the invention.

Embodiments of the invention provide a bone nail assembly that provides enhanced internal fixation of one or more long bones being treated (e.g., femur, tibia, fibula, ulna/radius, humerus), is particularly easy to deploy, or both. In addition, embodiments of the invention may facilitate alignment, stabilization, and/or fusion of a joint, such as an ankle or knee. Multiple examples of a bone nail assembly configured as an intramedullary nail assembly particularly suitable for fixing together the calcaneous, talus, and tibia in a human lower limb foot area are illustrated below.

Of course other configurations of the invention can provide a nail assembly for treating other bones. Also described below are exemplary nail assemblies configured for treating fractures in the proximal humerus and proximal femur. Other contemplated uses include, but are not limited to the proximal tibia and distal supracondylar femur. In addition, embodiments of the invention may be useful for treating simple and compound long bone shaft fractures, transverse fractures, fractures with bone loss, sub-trochanteric fractures, fractures distal to a hip implant, fractures proximal to a total knee arthoplasty, fractures distal to a total knee arthroplasty, severely comminuted, spiral, large oblique and segmental fractures, nonunions and malunions, polytrauma and multiple fractures, prophylactic nailing of impending pathologic fractures, reconstruction, following tumor resection and grafting, supracondylar fractures, including those with intra-articular extension, and/or acute bone lengthening and shortening. In addition, embodiments of the invention may also be useful for treating pseudoarthrosis and corrective osteotomies of long bones, reamed and unreamed applications, degeneration, deformity, or trauma of both the tibiotalar and talocalcaneal articulations in the hindfoot, tibiocalcaneal arthrodesis, combined arthrodesis of the ankle and sub-talar joints, avascular necrosis of the ankle and sub-talar joints, failed total ankle replacement with sub-talar intrusion, failed ankle arthrodesis with insufficient talar body, rheumatoid arthritis, severe deformity secondary to untreated talipes equinovarus or neuromuscular disease, severe pilon fractures with trauma to the sub-talar joint, and/or intramedullary knee arthrodesis.

Of course, the scope of the invention is not limited to any particular bone setting.

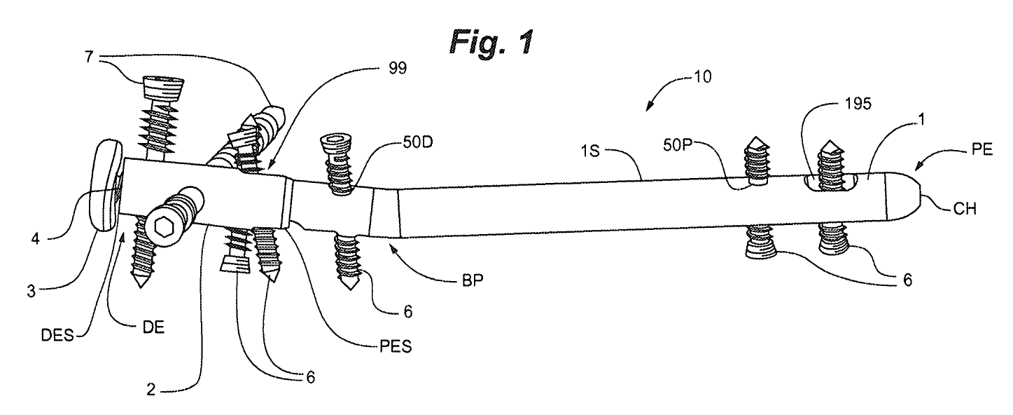

Turning to FIG. 1, one embodiment of the invention provides an intramedullary nail assembly 10 (also referred to as a “bone nail assembly”). The nail assembly 10 includes a bone nail 1 having a first end and a second end (e.g., proximal PE and distal DE ends, respectively) and an elongated nail shaft 1S extends between these ends. The terms proximal and distal are used herein with respect to a patient receiving the bone nail such that the proximal end PE is nearest the center of the patient and the distal end DE is farthest from the center of the patient. In some cases the terms first end and second end can also used to describe the ends of the nail, and either may correspond to “proximal” and/or “distal” as is appropriate for a particular configuration and/or anatomy. The nail shaft can be formed of any suitable material that does not generate rust and does not adversely affect bone marrow tissues. In some cases, the nail shaft is formed of metal, such as a titanium alloy. The particular material from which the bone nail is formed is not limiting to the invention.

The illustrated nail shaft is cannulated. Specifically, it has a cannulated hole CH along its length to accept a guide pin. For example, the cannulated hole can accept a guide wire and the nail shaft can be inserted through an incision over the guide wire using x-ray fluoroscopy for optimal positioning within a medullary canal. A cannulation, however, is by no means required. For example, in certain embodiments the nail shaft may be solid along part, or all of the length of the shaft.

In many cases, the nail shaft 1S has at least one transverse screw hole 50D adjacent the distal end DE of the shaft, and at least one transverse screw hole 50P adjacent the proximal end PE of the shaft. Each of these screw holes 50 passes through the nail shaft 1S (e.g., at an angle oblique to the nail's central axis) and is configured to receive a bone screw (e.g. cortical screw 6 or transition cortical/cancellous screw 7) for fixing the nail 1 within one or more desired bones. In the illustrated embodiment, there are a plurality of screw holes 50 adjacent the distal end DE of the nail shaft, and one screw hole 50P adjacent the proximal end PE of the nail shaft.

In addition, in some cases the nail shaft 1S also has one or more screw slots (e.g., slots 95, 195) that pass through the nail shaft 1S. As will be discussed further herein, the slot 95 proximate the distal end DE of the nail shaft can provide a measure of adjustability for compressing together one or more desired bones. In certain cases, the nail shaft 1S can include an optional proximal slot 195, which may be a dynamization slot. For example, after attempting to fuse an ankle without success, a surgeon may remove a bone screw from screw hole 50P and the tibia and instead insert a bone screw through the tibia and the slot 195. The tibia is thus provided with a limited range of axial movement, which may in some cases promote fusion in the lower limb foot area. Of course, the number and arrangement of screw holes and slots will vary in different embodiments and the scope of the invention is not limited in this regard.

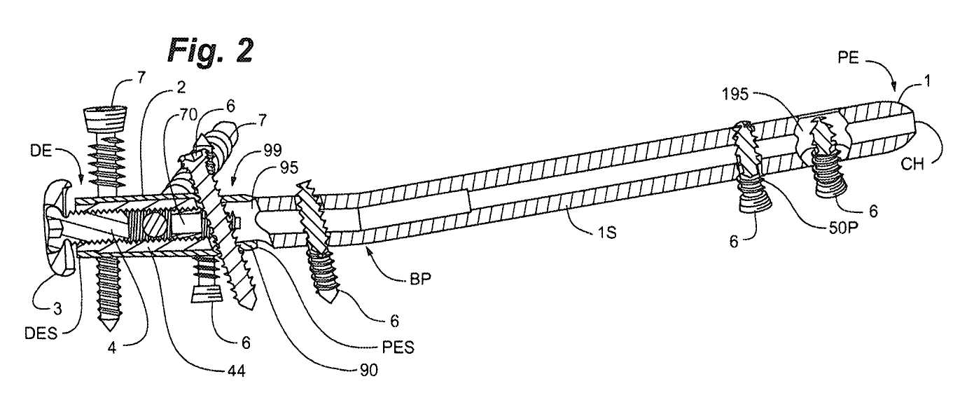

As shown in FIG. 2, in certain embodiments the nail shaft 1S preferably has a hollow internally threaded section 44 adjacent its distal end DE. In the illustrated embodiment, the internally threaded section 44 does not extend along the entire length of the nail shaft (although it is in communication with the cannulated hole CH, which extends from the hollow defined by internally threaded section 44 to the proximal end PE of the shaft 1S). Rather, it extends from the distal end DE and terminates well before reaching even the halfway point of the nail shaft's length. In the embodiment illustrated, the nail shaft has a bend point BP, and the internally threaded section 44 extends from the nail shaft's distal end DE toward its bend point, but terminates before reaching the bend point. People skilled in this technology area will appreciate that these details are merely optional, and will vary depending upon how the nail system is to be used. For example, in some cases, the nail shaft 1S may not include a hollow, internally threaded section adjacent its distal end DE and may instead merely include a continuation of the cannulated hole CH, or may be solid without a through hole. In addition, in some cases the cannulated hole CH may be threaded along its entire length.

In certain embodiments the internally threaded section 44 is adapted to threadedly receive one or more instruments extending out from the nail. For example, in some cases an instrument targeting arm with alignment holes is attached to the distal end of the nail via a threaded shaft received within the internally threaded section 44. The threaded section 44 thus fixes the targeting arm with respect to the nail shaft, which facilitates the targeting arm providing proper orientation, preparation, and alignment of the proper cannula for transverse angled drilling of bone screw holes into long bone cortical and cancellous bone. Thus, the targeting arm can provide proper alignment to the corresponding transverse and angled holes within the nail shaft 1S for insertion of one or more bone screws.

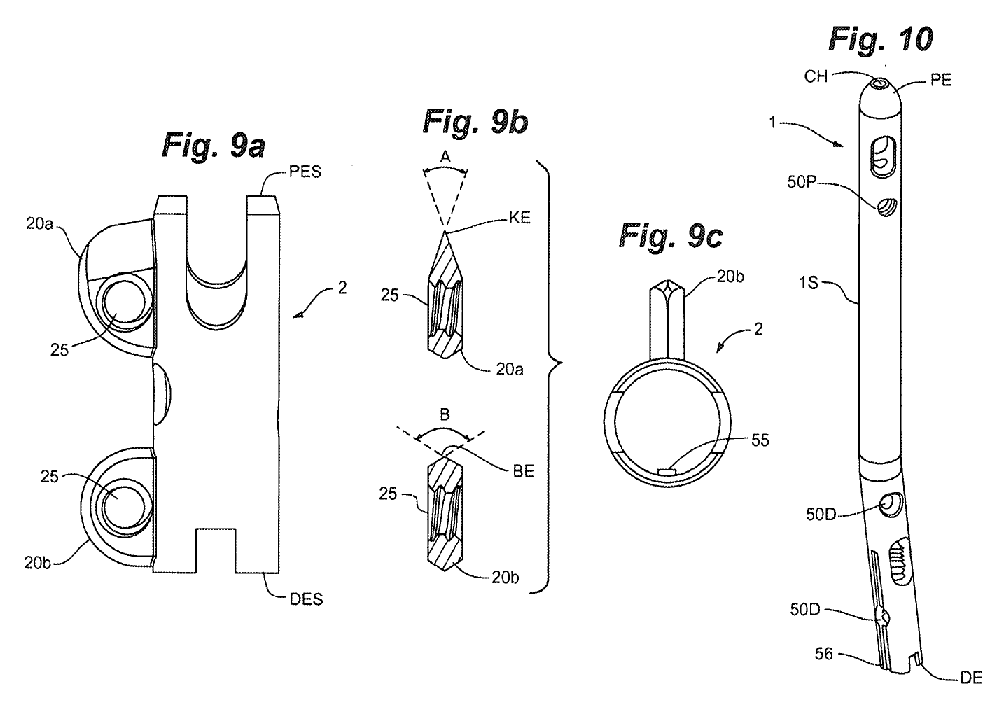

In the illustrated embodiment, an anchoring sleeve 2 surrounds the hollow internally threaded section 44 of the nail shaft. As shown in FIG. 2, the sleeve 2 defines an internal cavity configured to receive a section (e.g., a distal length) of the nail shaft 1. When the illustrated nail assembly is operatively assembled, the hollow internally threaded section 44 of the nail shaft 1S is nested concentrically within the anchoring sleeve 2. Preferably, the confronting surfaces of the sleeve 2 and the nail shaft 1S (i.e., the internal surface of the sleeve and the corresponding external surface of the nail shaft, which surfaces are carried against each other when the nail system is operatively assembled) are smooth surfaces that are carried slidably against each other, such that the sleeve is mounted slidably on the nail shaft (absent any restriction to such relative sliding, as would result once one or more transverse screws are anchored in place). Referring to FIGS. 9c and 10, in some cases the anchoring sleeve 2 includes a spline 55 that slides within a groove 56 in the external surface of the nail shaft 1S adjacent the distal end DE of the shaft. The spline 55 and groove 56 register or orient the anchoring sleeve 2 and the nail shaft 1S to prevent relative circumferential movement about the central axis of the nail shaft 1S.

It should be appreciated that multiple variations in sleeve placement are possible. While the illustrated embodiment shows the anchoring sleeve 2 surrounding the hollow internally threaded section 44 at the distal end of the nail shaft, in some cases the sleeve may be positioned about the nail shaft at the proximal end PE of the nail. In addition, depending upon the desired functionality and degree of fixation desired, more than one sleeve can be used. For example two sleeves can be placed on the nail, one at the distal end as illustrated and another at the proximal end. Of course, in cases in which the nail shaft is solid along its entire length, the sleeve is not positioned about a hollow internally threaded section, but is instead positioned about a solid section of the nail.

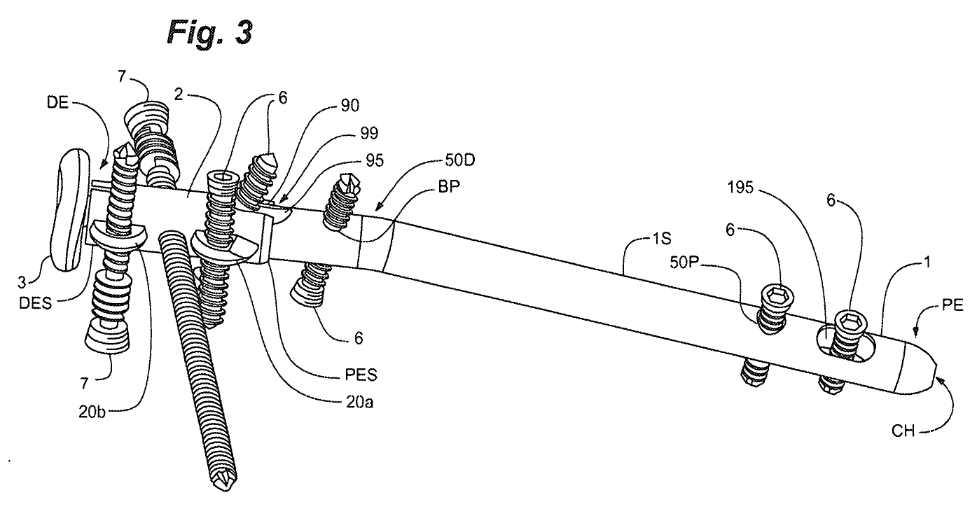

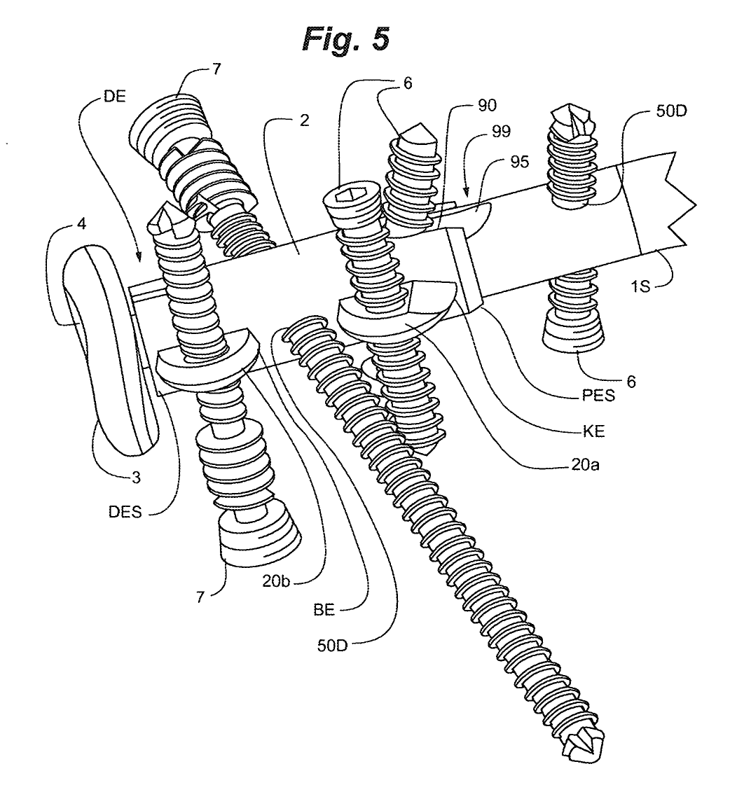

Referring to FIG. 5, it can be appreciated that one or more transverse screw openings 50 can optionally pass through both the anchoring sleeve 2 and the hollow internally threaded section 44 of the nail shaft 1S. In some cases a bone screw positioned within one or more of the transverse screw openings 50 engages both the nail shaft 1S and the anchoring sleeve 2, thus fixing the two components and preventing axial movement between the anchoring sleeve and the nail shaft. Referring to FIGS. 3, 4, 5, and 9a-9c, in a first aspect of the invention the nail assembly 10 is configured to provide particularly stable fixation of the nail 1 within one or more bones. In this aspect, the assembly 10 has at least one fin 20 that is provided with an internally threaded mount hole 25. The mount hole 25 is configured to receive a desired screw 6, 7 for fixing the nail 1 within one or more desired bones. The mount hole 25 is spaced radially from a central axis of the nail shaft 1 (and from a central axis of the sleeve 2). This creates a moment arm between the screw received in the mount hole and the central axis of the nail shaft. Arrangements of this nature provide resistance to micromotion of the nail assembly relative to the bone(s) in which it is fixed.

Preferably, the mount hole 25 on the fin 20 is spaced radially from the central axis of the nail shaft (and from a central axis of the sleeve) by at least 5 mm, at least 6 mm, at least 7 mm, at least 9 mm, or at least 11 mm. In one preferred embodiment, the sleeve 2 has an outer diameter of about 11 mm and the mount hole 25 on the fin 20 is spaced radially from the central axis of the nail shaft by about 8 mm. The reported dimension extends from the axis of the mount hole to the adjacent central axis of the nail shaft/sleeve. In one preferred embodiment, the mount hole 25 on the fin 20 is spaced radially from the exterior surface of the sleeve 2 by at least 2 mm, at least 2.5 mm, or at least 3 mm. In embodiments where multiple fins and/or multiple mount holes are provided, each mount hole can be spaced from the nail's central axis and/or the sleeve's exterior surface, as just described. As will be appreciated by those skilled in the present area of technology, the dimensions of the nail system will vary depending upon the particular application for which it is intended. Thus, the exemplary dimensions mentioned above are by no means limiting to the invention.

The mount hole 25 is internally threaded to provide for positive attachment between the fin 20 and the screw 6, 7 received in the mount hole. In some cases the screw 6, 7 is provided as a locking screw, which locks within the internally threaded mount hole 25. For example, an externally threaded screw “locks” within an internally threaded mount hole 25 in that the screw cannot freely translate through the mount hole without also turning. This provides increased resistance to the undesirable micromotion that has been observed with conventional nail systems. When multiple mount holes 25 are provided, each mount hole 25 preferably is internally threaded.

Thus, in the first aspect of the invention, the anchoring sleeve 2 has at least one outwardly projecting fin 20. Preferably, the fin is integral to (i.e., defined by the same body as) the anchoring sleeve. As noted above, the fin 20 has an internally threaded mount hole 25 configured to threadingly receive a screw 6, 7 for fixing the nail 1 within one or more desired bones, and this mount hole 25 is spaced radially from a central axis of the nail shaft, so as to create a moment arm between the screw and the central axis of the nail shaft. As already explained, this provides resistance to micromotion of the nail assembly relative to the bone(s) in which the nail system is fixed.

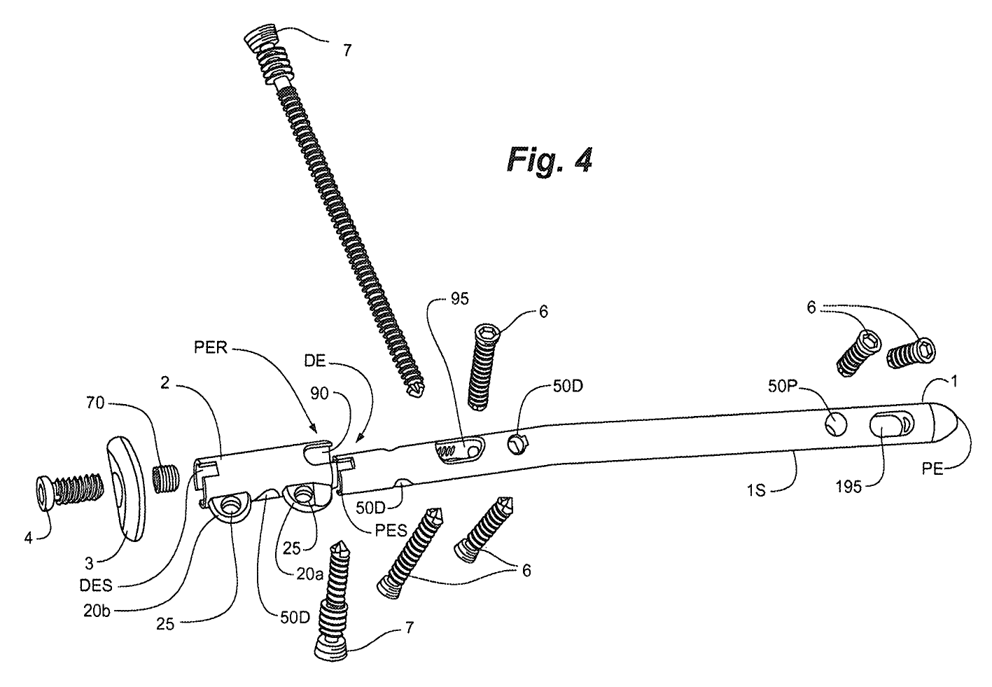

As shown in FIG. 4, the anchoring sleeve 2 has distal DES and proximal PES ends. The axial distance between these distal and proximal ends is the sleeve's axial length. In the illustrated embodiment, the nail system 10 has a mount hole 25 (defined by fin 20b) that is located within a distal ¼^{th} of the axial length from the sleeve's distal end. That is, the nail system has a fin 20b with a mount hole 25 located on the distal quarter of the sleeve. This is believed to contribute to the excellent fixation achieved by the present nail system. It is, however, not required in all embodiments.

As noted above, the anchoring sleeve 2 can advantageously have two fins 20a, 20b. Preferably, each fin is integral to (i.e. defined by) the sleeve. As is perhaps best seen in FIG. 5, a transverse screw opening 50D can optionally be provided so as to pass through both the anchoring sleeve 2 and a hollow internally threaded section 44 of the nail shaft 1S. In some cases this transverse screw opening can optionally be located axially between the two fins 20a, 20b. As is illustrated, the transverse screw opening 50D is oriented with respect to the mount holes 25 in the fins 20a, 20b, such that the bone screw 7 extending through the transverse screw opening 50D generally crosses the two screws fixed to the fins 20a, 20b. For example, the transverse screw hole and the mount holes may be generally or substantially perpendicular to each other, or oriented at another crossing angle. This cross-screw orientation is believed to contribute to the nail system's particularly good fixation, although it is not strictly required. For example, in some cases the mount holes 25 in the fins 20a, 20b are oriented with respect to one or more screw holes in the nail shaft 1S such that one or more screws extending through the nail shaft are in a parallel configuration with the screws extending through the mount holes 25. In still further variations, the nail assembly may be configured with only minimal or no cross-screw orientations. Other variations are also contemplated, including a crossing orientation between the two fins 20a, 20b and mount holes 25 (rather than the illustrated parallel configuration) and/or between all of the fins/mount holes and the bone screw 7 extending through the transverse hole 50D.

Each fin 20 may have a generally flat, plate-like configuration. In the illustrated embodiment, each of the two fins 20a, 20b has a generally circular or half-circular shape, although the fin shape can be varied. The illustrated fins 20a, 20b are arranged such that they both lie in a common plane, which is a radial plane (i.e., a plane parallel to, and lying on, a central axis of the sleeve). This, however, need not always be the case. For example, in some embodiments two or more fins may be angularly spaced about the central axis of the sleeve (i.e., in different radial planes), as will be discussed further herein.

In the embodiment of FIG. 5, and also shown in FIGS. 9a-9c, the first fin 20a has a proximal knife edge KE facing the first/proximal end of the sleeve and nail, while the second fin 20b has a generally blunt proximal edge BE facing the same direction. For example, in some cases the surfaces of the knife edge may be formed at an angle A less than 70 degrees, less than 50 degrees, or less than 30 degrees. In one preferred embodiment the surfaces of the knife edge are formed at an angle A of about 40 degrees. In certain cases the surfaces of the blunt edge may be formed at an angle B more than 60 degrees, more than 100 degrees, or more than 140 degrees, but less than 180 degrees. In one preferred embodiment the surfaces of the blunt edge are formed at an angle B of about 120 degrees. In other embodiments, though, this knife edge KE may be omitted, or a knife edge may be added to the second fin 20b.

Reference is now made to FIG. 4, which depicts an embodiment wherein the anchoring sleeve 2 includes two fins 20a, 20b spaced axially apart along a length of the sleeve. Here, a first 20a of the fins is located closer to a proximal end PES of the sleeve 2 than is a second 20b of the fins. In FIG. 5, it can be appreciated that each of the two fins 20a, 20b has an internally threaded mount hole 25. Additionally or alternatively, two screws 6, 7 of different lengths (but optionally of the same diameter) are mounted respectively in these two mount holes 25. As is shown, the mount holes 25 can optionally receive screws 6, 7 from opposite directions. While these details are not required, such arrangements are believed to contribute favorably to achieving exceptional internal fixation performance. In addition, the mount holes 25 within the fins 20a, 20b may be oriented in a parallel configuration as shown, or in an angled or skew configuration if a degree of cross-screw fixation is desired from the bone screws 6, 7.

In the illustrated embodiment, a proximal end region PER (see FIG. 4) of the anchoring sleeve 2 defines a slot 90 that is open through a proximal end PES of the sleeve. A corresponding slot 95 is defined by the nail shaft 1S, such that when the sleeve 2 is operatively mounted on the nail shaft, these slots 90, 95 are aligned to define a slot-shaped transverse screw passage 99 (see FIGS. 2 and 3). FIGS. 2 and 5 depict the manner in which a transverse screw 6 can be mounted in this passage 99. The slots 90, 95 are configured such that when a bone screw 6 extends through the slot-shaped passage 99, the anchoring sleeve 2 and the nail shaft 1S have a desired amount of freedom to move axially relative to the bone screw 6. When the proximal end PE of the nail shaft 1S is fixed in bone with a bone screw 6 extending through the proximal transverse screw hole 50P, the axial movement of the shaft and sleeve relative to the transverse screw 6 in the slot-shaped passage 99 allows the nail system to compress the bone(s) being treated.

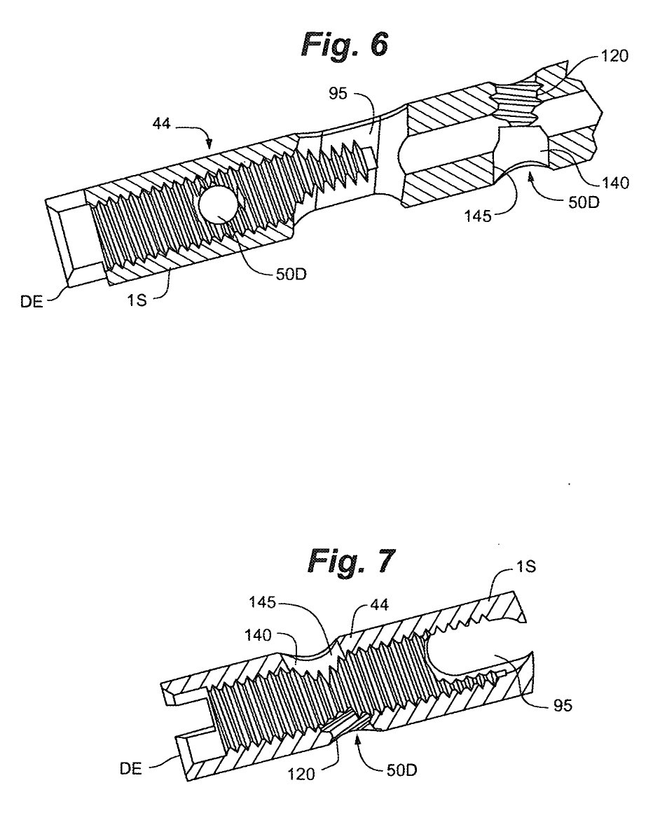

In the illustrated embodiment, the slot 95 in the nail shaft 1S is bounded on two confronting sides by threaded internal wall sections of the nail shaft. This is best appreciated with reference to FIGS. 2 and 6.

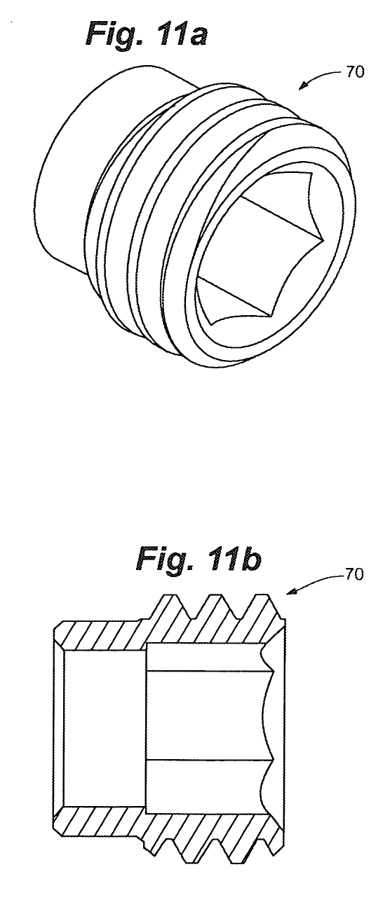

FIGS. 2 and 4 illustrate two alternative methods for compressing multiple bones or bone fragments with the nail assembly 10. According to one method, the illustrated nail system includes an internal compression screw 70 configured to be threadingly mounted inside the internally threaded section 44 of the nail shaft 1S. FIGS. 11a-11b provide detailed views of the internal compression screw 70. Returning to FIGS. 2 and 4, the internal compression screw 70 can be advanced through the hollow internally threaded section against the screw 6 that is mounted within slot passage 99. Since the bone screw 6 is fixed within the bone surrounding slot passage 99, advancing the compression screw 70 against the bone screw 6 advantageously pulls the first end PE of the nail shaft toward the bone screw 6 to provide bone compression. For example, when located in an ankle position, the screw 6 through the slot passage 99 can be fixed within the calcaneous, while the screw 6 through the proximal transverse hole 50P can be fixed within the tibia. Advancing the internal compression screw 70 pulls the tibia toward the calcaneous to compress the tibia, talus, and calcaneous for fusion.

Continuing with reference to FIGS. 2 and 4, according to an alternative embodiment of the invention, an external axial compression screw 4 is threadingly received in the hollow internally threaded section 44 of the nail shaft 1S, such that the compression screw and the hollow internally threaded section are threadingly engaged, while the anchoring sleeve 2 is mounted slidably on the nail shaft. The illustrated axial compression screw 4 carries a washer member 3. As shown in FIG. 2, the axial compression screw 4 and the washer member 3 preferably are separate bodies that can be disassembled from each other. The compression screw 4 and the washer member 3 are configured such that in response to a relative rotation of the axial compression screw and the nail shaft, the washer member bears against one or more desired bones proximate the distal end DE of the nail shaft and sleeve DES so as to pull the first end of the nail shaft toward the washer member 3 to compress multiple bones or pieces of fractured bone together.

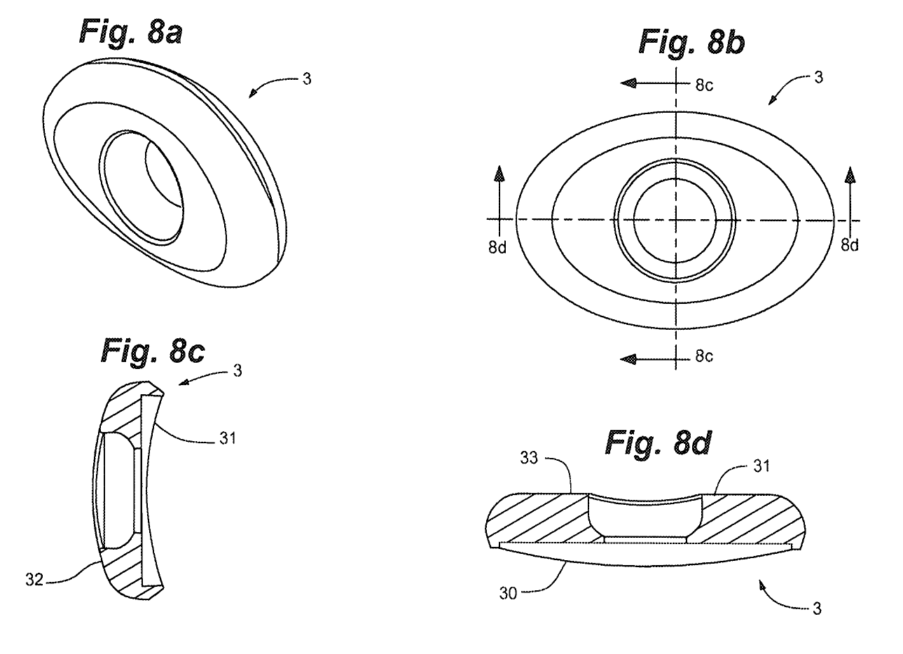

FIGS. 8a-8d provide various views of the washer member 3. The washer member 3 and the screw 4 provide a mechanism for fixing the nail assembly within desired bones while also compressing together the desired bones. The washer member 3 and screw 4 can be used in addition to or instead of multiple locking screws passing through the distal end of the nail shaft 1S (e.g., instead of bone screws 6, 7 extending through holes 50D, passage 99, and/or mount holes 25). In addition, although FIGS. 2 and 4 illustrate both the external compression screw/washer 4/3 and the internal compression screw 70, it is contemplated that in a typical setting only one may be used.

In certain embodiments the washer member 3 is an implantable washer, configured to remain with the nail assembly within the bone(s) and/or surrounding tissue. An implantable compression washer 3 and screw 4 provide a more easily deployable nail assembly in that the bone nail can be fixed within the bone(s) with the single procedural step of inserting and tightening the compression screw 4 such that the washer member 3 presses up against the bone(s) and the distal end DES of the sleeve 2. This provides a simpler method of deploying a nail assembly than a conventional method in which a tool is used to compress the bone(s), bone screws are inserted to fix the bone(s) in the compressed state, and then the tool is removed.

FIGS. 8a-8d illustrate a washer member 3 particularly suitable for implantation with a nail assembly configured to fix together the calcaneous, talus, and tibia of the ankle. As shown, the washer member 3 is provided with a rounded, oval shape designed to contact the calcaneous anterior to the lateral and medial processes. A bottom surface is provided with transverse convex 30 and concave 31 curvatures forming a saddle contoured bottom surface to more closely align with that portion of the calcaneous. In addition, a top surface of the washer member 3 is provided with a similar saddle contour with transverse convex 32 and concave 33 curvatures to more naturally follow the contour of the human foot at that portion of the calcaneous.

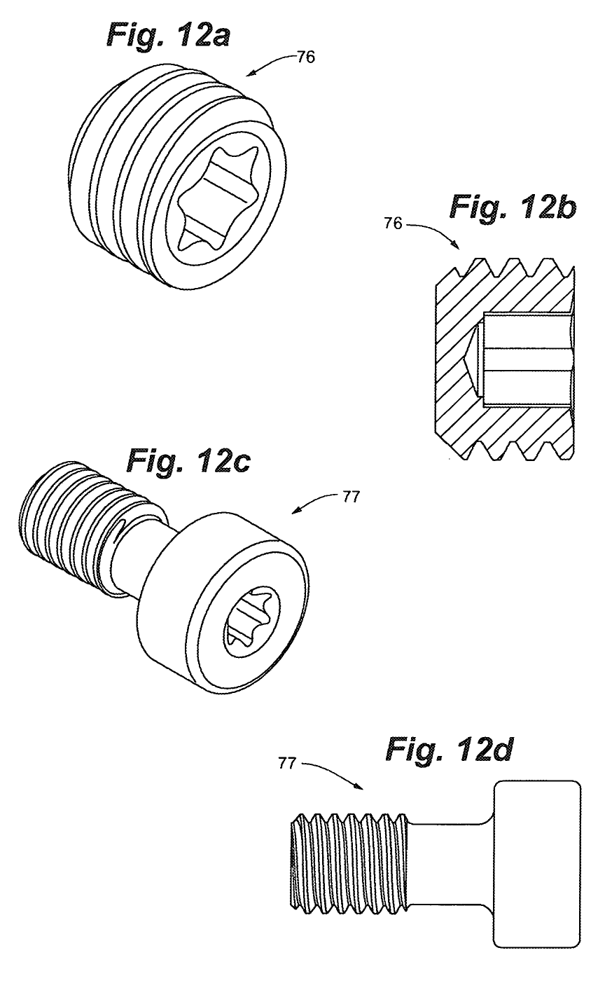

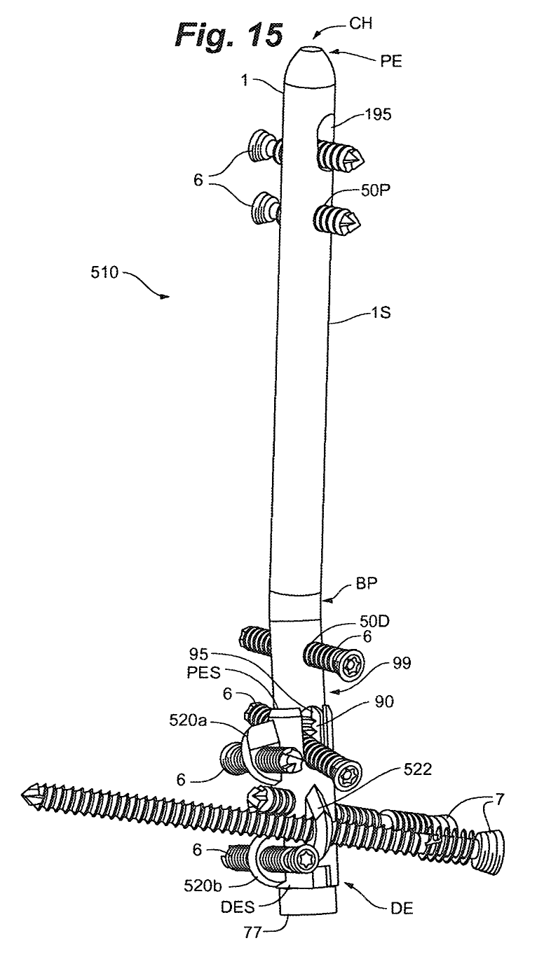

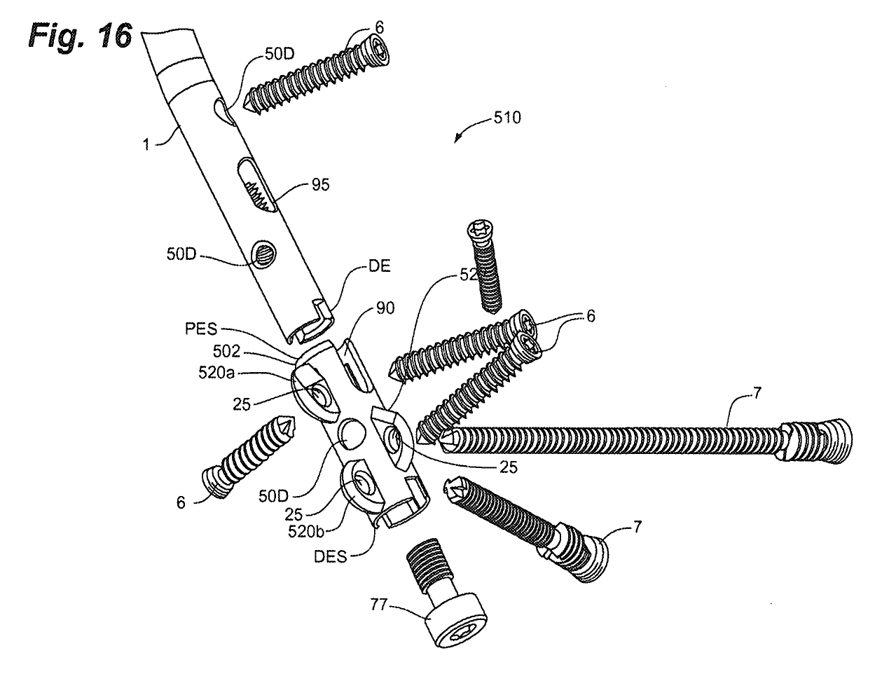

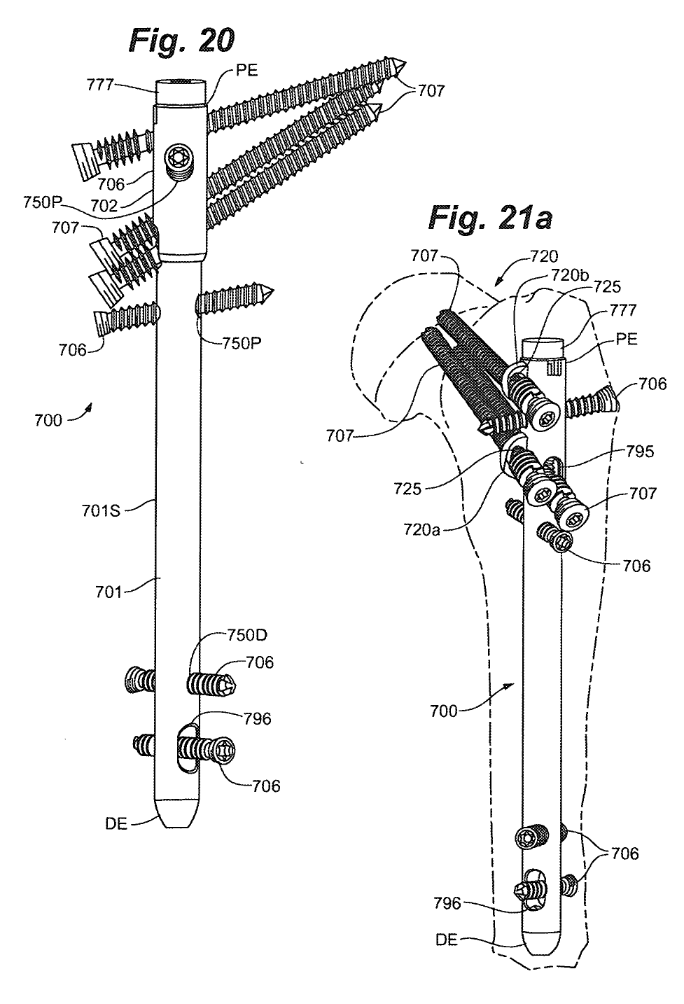

Referring to FIGS. 12a-12d, views of optional end caps 76, 77 are provided. In some cases transverse screws may provide the sole fixation means and one of the implantable end caps 76, 77 is received in the distal end DE of the nail shaft instead of the screw 4 and the external washer member 3 shown in FIGS. 1-5. For example, FIGS. 15 and 16 illustrate one embodiment employing the end cap 77. The end cap 77 is also shown in use in the embodiments depicted in FIGS. 20, 23, and 26a-26c.

The end caps 76, 77 are useful for closing the end of the nail so that tissue does not grow into the open end of the nail. This can be especially helpful in the case that the nail needs to be extracted at a later time. The end cap 76, 77 can be removed and an extraction instrument can be threaded into the still open and of the nail. The end cap 77 is also useful for providing further support within the end of a long bone implanted with the nail. For example, the head of the end cap 77 may be approximately the same size as the diameter of the nail, which allows the end cap to effectively extend the length of the nail, providing additional support within the canal area at the end of a bone.

In a second aspect of the invention, the nail assembly 10 is provided with a novel screw hole configuration that makes it particularly easy to deploy the bone nail system within one or more bones to be treated. As discussed above with respect to FIGS. 1-4, one bone nail assembly 10 includes a bone nail 1 having distal DE and proximal PE ends and an elongated nail shaft 1S extending between those ends. At least one screw hole 50 passes through the nail shaft 1S and is configured to receive a screw 6, 7 for fixing the nail 1 within one or more bones. Referring to FIGS. 6 and 7, in certain embodiments the screw hole 50 comprises a threaded first portion 120 and a non-threaded second portion 140. The non-threaded second portion 140 is configured to receive a screw at different approach angles and guide such screw to the threaded first portion 120. In some cases the threaded first portion 120 is configured to lock the screw 6, 7 within the hole 50.

The non-threaded hole portion 140 is bounded by a non-threaded (e.g., smooth) wall 145, which preferably is defined by the nail shaft 1S. In the illustrated embodiment, the nail shaft 1S itself defines an entirety of an interior wall 145 that defines the entire passage for a screw passing through the non-threaded hole portion. The nail shaft wall 145 defining the non-threaded hole portion 140 preferably delineates a screw passage (or “path”) having a diameter greater than or equal to (or at least substantially equal to) a maximum diameter of the threaded portion 120 (this maximum diameter may be the diameter of the thread portions of the threaded bore 120). This can be seen in FIG. 6. The non-threaded portion 140 of the screw hole can optionally be a counter bore. Preferably, the non-threaded portion 140 of the screw hole 50 is devoid of an insert occupying any portion of the screw hole.

Typically, a plurality of screw holes 50 pass through the nail shaft 1S and are each configured to receive a screw 6, 7 for fixing the nail 1 within one or more desired bones. Preferably, at least two of these screw holes 50 comprise a threaded first portion 120 and a non-threaded second portion 140 of the nature described above (e.g., bounded by a smooth wall of the nail shaft, configured to receive a screw at different approach angles and guide such screw to the threaded first portion, and being devoid of an insert occupying any portion of the screw hole).

In some embodiments, at least one such screw hole 50 passes through a hollow internally threaded section 44 of the nail shaft 1S. This is perhaps best appreciated with reference to FIGS. 5 and 7. Preferably, the hollow internally threaded section 44 of the nail shaft 1S is adjacent the distal end DE of the nail 1, as already discussed.

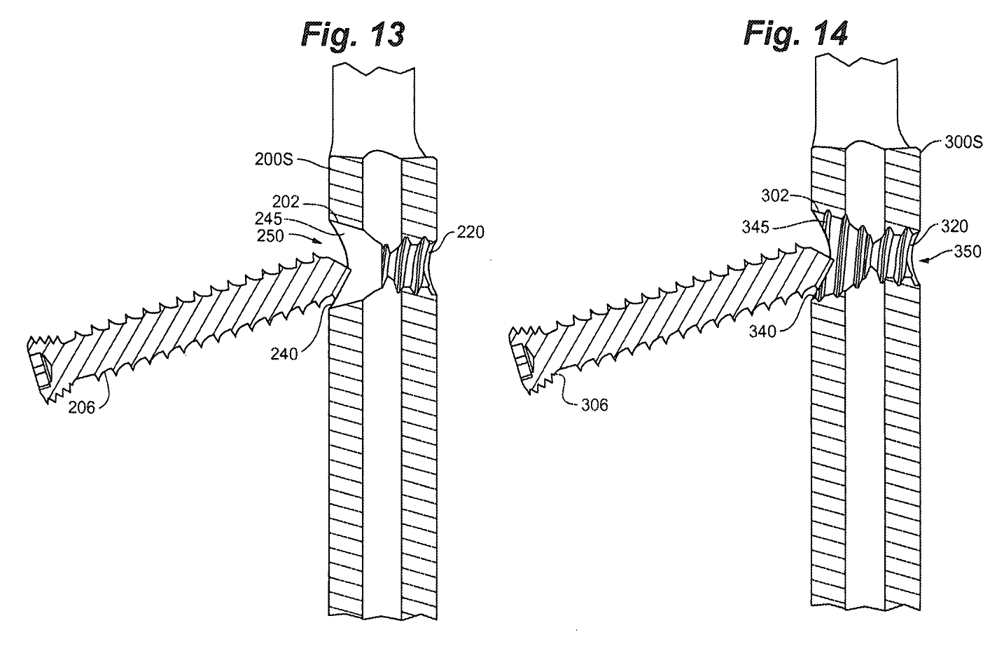

Referring again to FIG. 6, it can be seen that in some cases both the threaded first portion 120 and the non-threaded second portion 140 are in alignment, with substantially straight walls extending through the nail shaft 1S. Turning to FIG. 13, another embodiment of the invention provides a novel screw hole configuration including a screw hole 250 within a nail shaft 200S. The screw hole 250 comprises a substantially straight threaded first portion 220 and an angled non-threaded second portion 240. The angled second portion 240 is configured to receive a screw 206 at different approach angles and guide such screw to the substantially straight threaded first portion 120. In some cases the threaded first portion 220 is configured to lock the screw 206 within the hole 250.

The angled hole portion 240 is bounded by a non-threaded (e.g., smooth) wall 245, which defines the angle of the second portion and preferably is defined by the nail shaft 200S. According to some embodiments, the second portion has an angle of between about 30 degrees and 45 degrees relative to a central axis of the hole 250. In the illustrated embodiment, the nail shaft 200S itself defines an entirety of an interior wall 245 that defines the entire passage for a screw passing through the angled hole portion. The nail shaft wall 245 defining the angled hole portion 240 preferably delineates a screw passage (or “path”), with the wall angled inward toward the center of the screw hole 250 from an exterior surface 202 of the nail shaft 200S to the threaded first portion 220 of the screw hole 250. The angled portion 240 of the screw hole can optionally be a counter bore. Preferably, the angled, non-threaded portion 240 of the screw hole 250 is devoid of an insert occupying any portion of the screw hole.

Turning to FIG. 14, another embodiment of the invention provides a novel screw hole configuration including a screw hole 350 within a nail shaft 300S. The screw hole 350 comprises a substantially straight threaded first portion 320 and an angled threaded second portion 340. The screw hole 350 and first and second portions 320, 340 are similar in many respects to the example illustrated in FIG. 13, with the additional feature of a threaded, angled wall 345 of the second portion 340. The addition of threading to the second portion wall 345 can facilitate insertion of the screw 306 because the threading can catch the external threading of the screw to further guide and advance the screw through the hole 350 into the first portion 320 of the hole. Certain embodiments of the invention combine two or more aspects described herein. According to one embodiment, the nail assembly can have both the threaded mount hole 25/fin 20 feature and one or more screw holes with separate threaded and non-threaded portions and/or with separate substantially straight and angled portions. In these embodiments, the nail assembly can have any or all of the various characteristics and parameters described above in connection with the threaded mount hole 25/fin 20 feature, in combination with any or all of the various characteristics and parameters described above with respect to the novel screw holes.

Embodiments of the invention also provide methods for treating bones. According to one embodiment, a method of implanting the bone nail assembly 10 of FIG. 1 in an ankle includes inserting the nail 1 through the calcaneous, through the talus, and into the medullary canal of the tibia. The proximal end PE of the nail 1 is secured in the tibia with the bone screw 6 advanced through the proximal transverse screw hole 50P. Another bone screw 6 is advanced through the calcaneous into the slot-shaped transverse screw passage 99. In certain cases the internal compression screw 70 is then advanced through the hollow internally threaded section against the screw 6 that is mounted within slot passage 99. Advancing the compression screw 70 against the bone screw 6 pulls the first end PE of the nail shaft toward the bone screw 6 to compress the tibia, talus, and calcaneous for fusion. After the desired amount of compression is applied, additional bone screws 6, 7 are advanced through the calcaneous and talus, and through distal transverse screw holes 50D in order to secure the bones in their compressed state. In addition, bone screws 6 are advanced through the calcaneous into fin mount holes 25 in order to further stabilize the bone nail assembly. After successful implantation, an end cap such as one of the end caps 76, 77 shown in FIGS. 12a-12d can be inserted into the distal end of the nail 1 to seal the nail from the exterior environment.

According to another embodiment, a method of implanting the bone nail assembly 10 of FIG. 1 in an ankle includes inserting the nail 1 through the calcaneous, through the talus, and into the medullary canal of the tibia. The proximal end PE of the nail 1 is secured in the tibia with the bone screw 6 advanced through the proximal transverse screw hole 50P. In certain cases the external compression screw 4 and washer are advanced into the hollow internally threaded section against the portion of the calcaneous surrounding the distal end of the nail and the sleeve. Advancing the screw 4 and washer 3 against the bone pulls the first end PE of the nail shaft toward the washer 3 to compress the tibia, talus, and calcaneous for fusion. In certain embodiments the screw 4 and washer 3 are left implanted in the foot and no additional bone screws are needed to fix the ankle bones. In some cases, though, one or more additional bone screws 6, 7 are advanced through the calcaneous and talus, and through distal transverse screw holes 50D in order to secure the bones in their compressed state. In addition, in some cases bone screws 6 are advanced through the calcaneous into fin mount holes 25 in order to further stabilize the bone nail assembly.

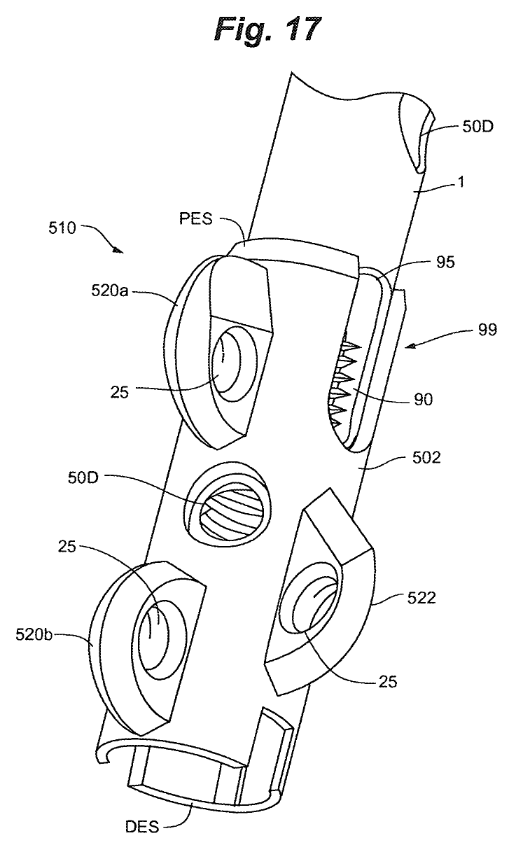

FIGS. 15-18 provide various detailed views of an intramedullary bone nail assembly 510 according to another embodiment of the invention. The nail assembly 500 includes the bone nail 1 having a first end and a second end (e.g., proximal PE and distal DE ends, respectively) and an elongated nail shaft 1S extends between these ends. The nail 1 is essentially as described above with respect to FIGS. 1-7, and thus further description is omitted here.

In the illustrated embodiment, an anchoring sleeve 502 surrounds the hollow internally threaded section 44 of the nail shaft 1S. The anchoring sleeve 502 is similar to the anchoring sleeve 2 described above with respect to FIGS. 1-7, but also provides additional stability and fixation for the nail assembly 510 within surrounding bone with the addition of a third fin extending from the sleeve 502. Accordingly, the anchoring sleeve 502 includes two fins 520a, 520b spaced axially apart along a length of the sleeve 502. As illustrated, one fin 520a is located closer to a proximal end PES of the sleeve 502, while the other fin 520b is located closer to a distal end DES of the sleeve.

The sleeve 502 also includes a third fin 522, which is angularly spaced about a central axis of the sleeve 502 from the first and second fins 520a, 520b. In other words, the third fin 522 is located in a different radial plane about the central sleeve axis from the first and second fins. This third fin 522 can be configured with dimensions and a shape similar to fins described herein above. The multiple fins may be separated by any desired angular spacing, which may vary depending upon the number of desired fins and the anatomy in which the nail and sleeve are placed. Thus, the anchoring sleeve 502 has at least two fins (520a, 520b) spaced axially apart along the length of the sleeve and at least two fins (520a, 522 or 520b, 522) angularly spaced about the central axis.

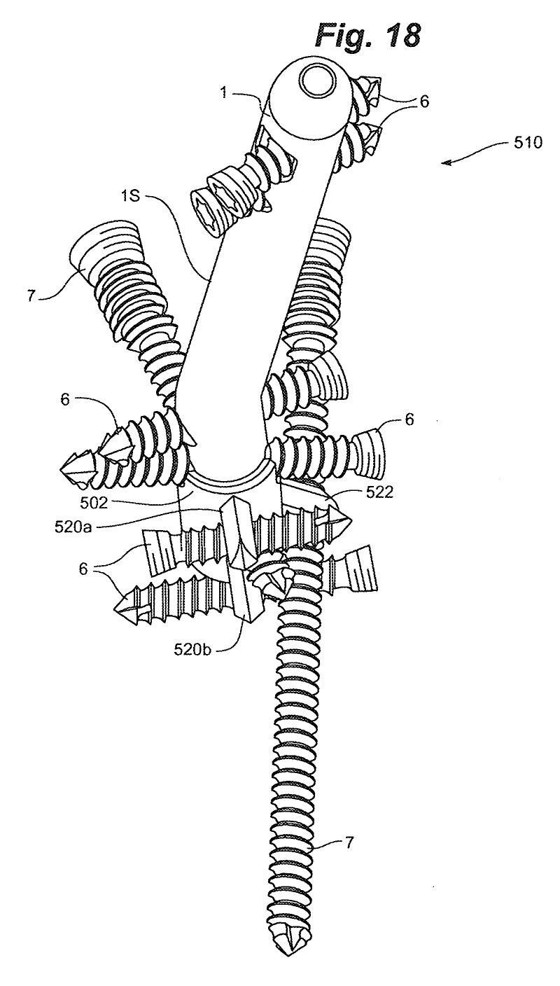

As described above, each of the fins is provided with an internally threaded mount hole 25. The mount holes 25 are configured to receive bone screws 6, 7 for fixing the nail 1 within one or more desired bones. In each fin, the mount hole 25 is spaced radially from a central axis of the nail shaft 1 (and from the central axis of the sleeve 502). This creates a moment arm between the screw received in the mount hole and the central axis of the nail shaft. The arrangement of multiple fins and screws spaced along the length and angularly about the central axis of the sleeve provides improved resistance to micromotion of the nail assembly 510 relative to the bone(s) in which it is fixed. In addition, the multiple fins and mount holes arranged at different angular positions about the axis of the sleeve (and with optionally different orientations) contributes to enhanced cross-screw fixation between the various screws 6, 7 as depicted. This is depicted in FIGS. 15 and 18.

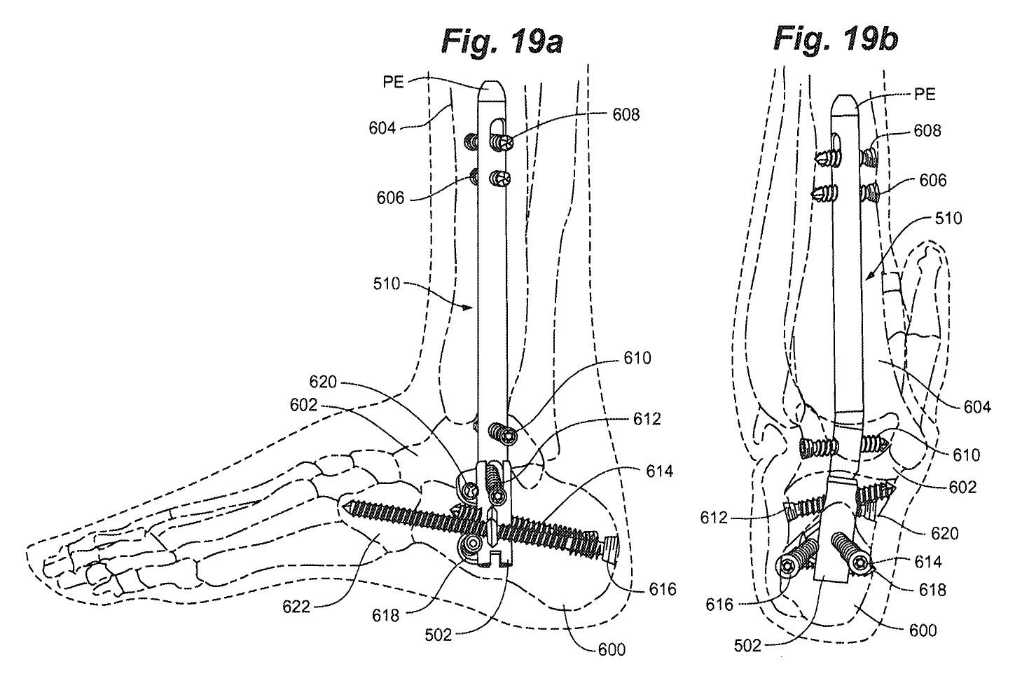

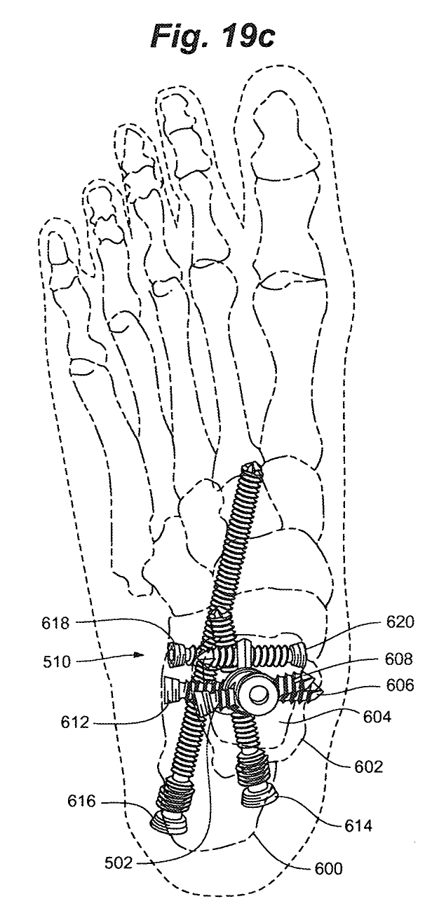

FIGS. 19a-19c depict the bone nail assembly 510 implanted within a human ankle according to an embodiment of the invention. As illustrated, the nail 1 is implanted through the calcaneous 600, through the talus 602, and into the medullary canal of the tibia 604. The proximal end PE of the nail 1 is secured in the tibia 604 with a first bone screw 606 and optionally a second bone screw 608. A third bone screw 610 secures the nail 1 in the talus 602. Finally, a number of bone screws 612, 614, 616, 618, and 620 secure the distal end DE of the nail 1 with the attached anchoring sleeve 502 within the calcaneous 600. One or more screws may also enter the navicular 622 and/or other adjacent foot bones.

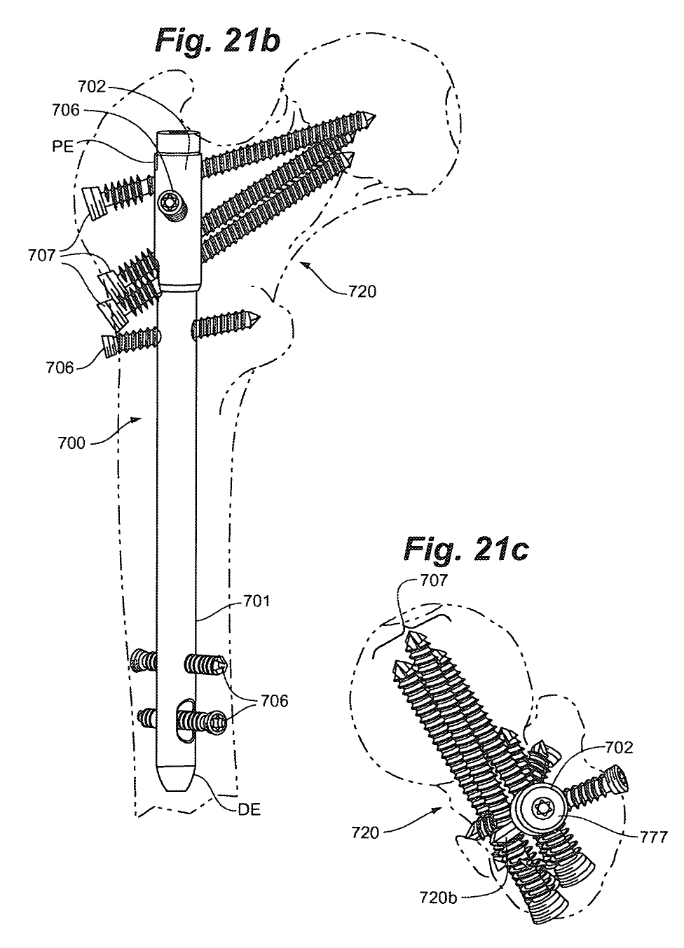

FIG. 20 is a side view of another intramedullary nail assembly 700 in accordance with certain embodiments of the present invention. As shown in FIGS. 21a-21c, the nail assembly 700 is particularly suited for proximal femoral implantation to facilitate healing of a broken femoral neck 720. The nail assembly 700 includes a bone nail 701 having a first end and a second end (e.g., distal DE and proximal PE ends, respectively) and an elongated nail shaft 701S extends between these ends.

In many cases, the nail shaft 701S has at least one transverse screw hole 750D adjacent the distal end DE of the shaft, and at least one transverse screw hole 750P adjacent the proximal end PE of the shaft. Each of these screw holes 750 passes through the nail shaft 701S (e.g., at an angle oblique to the nail's central axis) and is configured to receive a screw 706 for fixing the nail 701 within the proximal femur and femoral neck. In the illustrated embodiment, there are a plurality of screw holes 750P adjacent the proximal end PE of the nail shaft, and one screw hole 750D adjacent the distal end DE of the nail shaft.

In addition, in some cases the nail shaft 701S also has one or more screw slots (e.g., slots 795, 796) that pass through the nail shaft 701S. Of course, the number and arrangement of screw holes and slots will vary in different embodiments and the scope of the invention is not limited in this regard.

In the illustrated embodiment, an anchoring sleeve 702 surrounds the proximal end of the nail shaft (which may also be a hollow internally threaded section as described in previous embodiments). The sleeve 702 provides similar functionality to embodiments described elsewhere herein. In particular, the sleeve 702 has two fins 720a, 720b that are each provided with an internally threaded mount hole 725. The mount hole 725 is configured to receive a desired screw 707 for fixing the nail 701 within one or more desired bones.

As discussed in more detail in previous embodiments, the mount holes 725 are spaced radially from a central axis of the nail shaft 701 (and from a central axis of the sleeve 702). This creates a moment arm between the screws received in the mount holes and the central axis of the nail shaft. The mount holes 725 are preferably internally threaded to provide for positive attachment between the fins and the screws 707 received in the mount holes. Arrangements of this nature provide resistance to micromotion of the nail assembly relative to the bone(s) in which it is fixed. In some cases an end cap 777 is provided for closing the proximal end of the nail 701 after implantation.

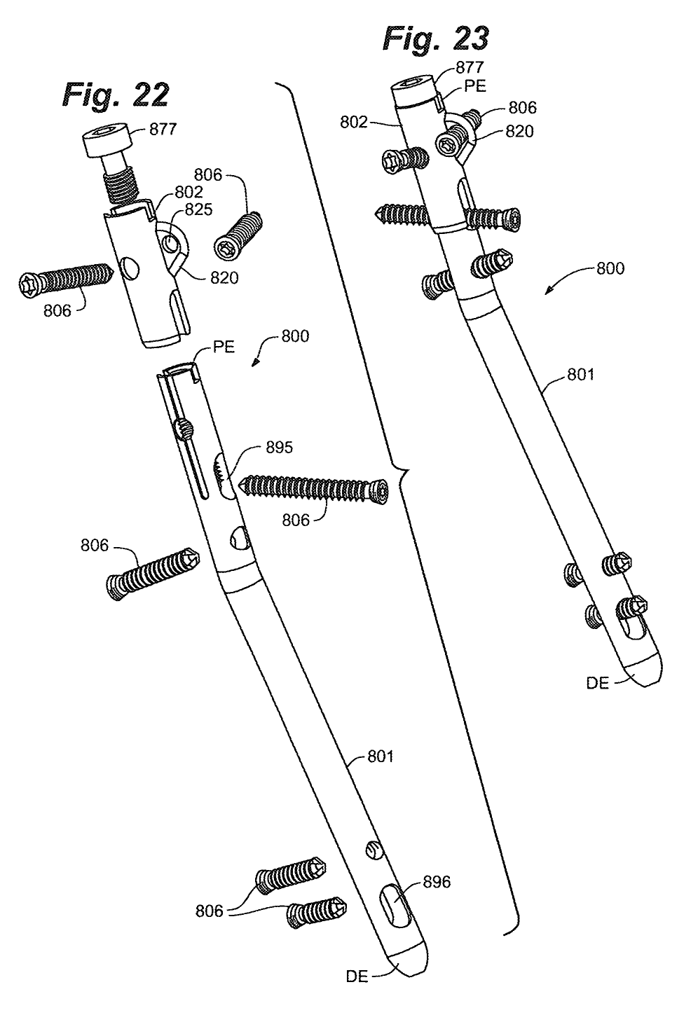

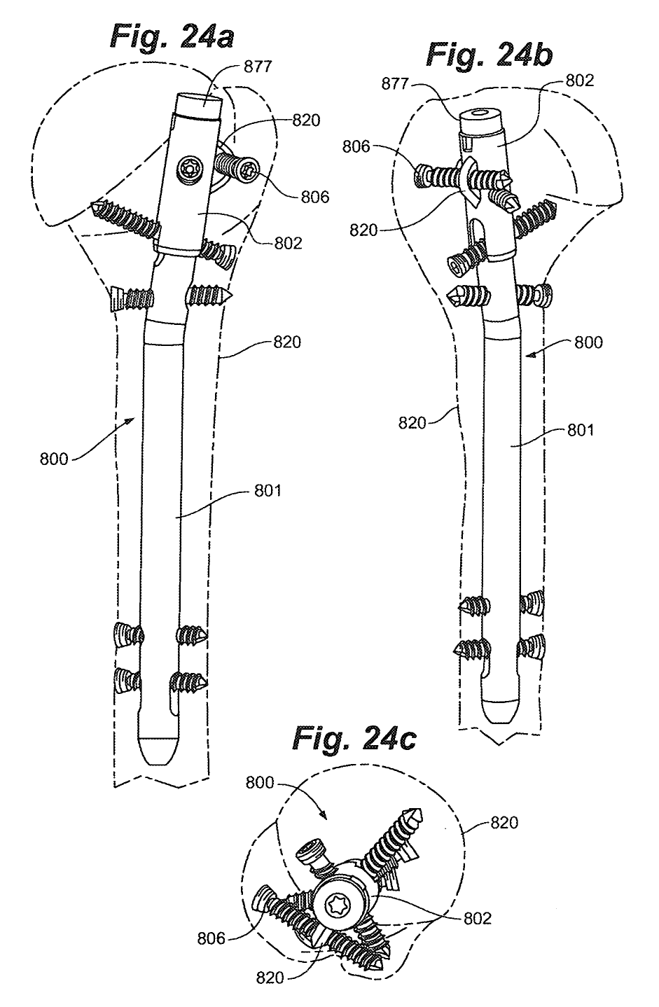

FIGS. 22 and 23 are views of another intramedullary nail assembly 800 in accordance with certain embodiments of the present invention. As shown in FIGS. 24a-24c, the nail assembly 800 is particularly suited for proximal humerus implantation to facilitate healing of a broken humerus 820. The nail assembly 800 includes a bone nail 801 having a first end and a second end (e.g., distal DE and proximal PE ends, respectively) and an elongated nail shaft 801S extends between these ends.

In many cases, the nail shaft 801S has at least one transverse screw hole 850D adjacent the distal end DE of the shaft, and at least one transverse screw hole 850P adjacent the proximal end PE of the shaft. Each of these screw holes 850 passes through the nail shaft 801S (e.g., at an angle oblique to the nail's central axis) and is configured to receive a screw 806 for fixing the nail 801 within the proximal humerus. In the illustrated embodiment, there are a plurality of screw holes 850P adjacent the proximal end PE of the nail shaft, and one screw hole 850D adjacent the distal end DE of the nail shaft.

In addition, in some cases the nail shaft 801S also has one or more screw slots (e.g., slots 895, 896) that pass through the nail shaft 801S. Of course, the number and arrangement of screw holes and slots will vary in different embodiments and the scope of the invention is not limited in this regard.

In the illustrated embodiment, an anchoring sleeve 802 surrounds the proximal end of the nail shaft (which may also be a hollow internally threaded section as described in previous embodiments). The sleeve 802 provides similar functionality to embodiments described elsewhere herein. In particular, the sleeve 802 has a fin 820 that is provided with an internally threaded mount hole 825. The mount hole 825 is configured to receive a desired screw 806 for fixing the nail 801 within the humerus.

As discussed in more detail in previous embodiments, the mount hole 825 is spaced radially from a central axis of the nail shaft 801 (and from a central axis of the sleeve 802). This creates a moment arm between the screw received in the mount hole and the central axis of the nail shaft. The mount hole 825 is preferably internally threaded to provide for positive attachment between the fin and the screw 806 received in the mount hole. Arrangements of this nature provide resistance to micromotion of the nail assembly relative to the bone(s) in which it is fixed. In some cases an end cap 877 is provided for closing the proximal end of the nail 801 after implantation.

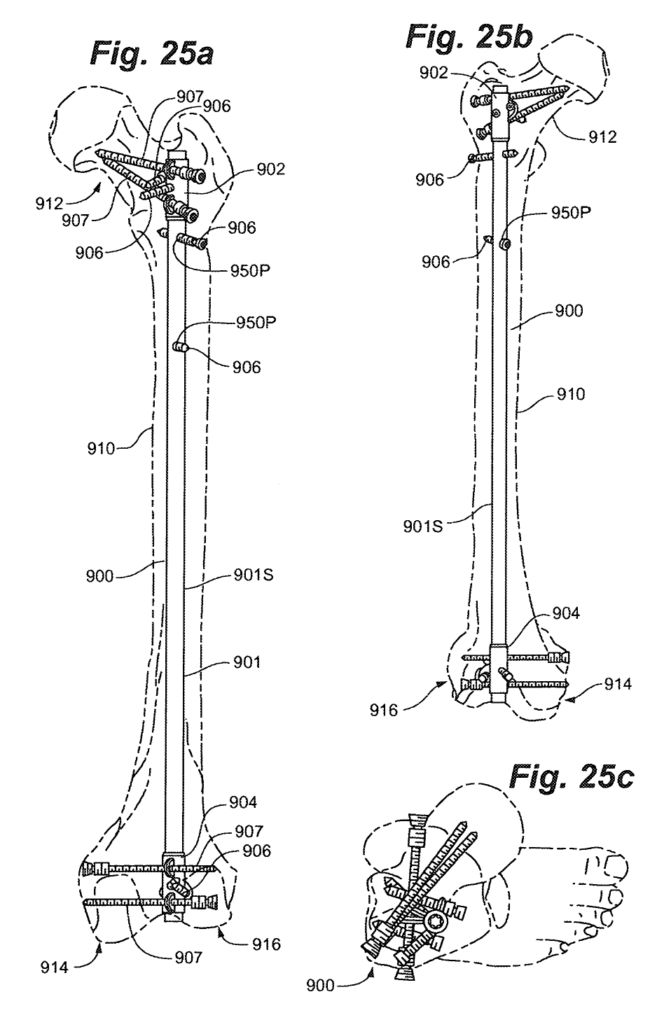

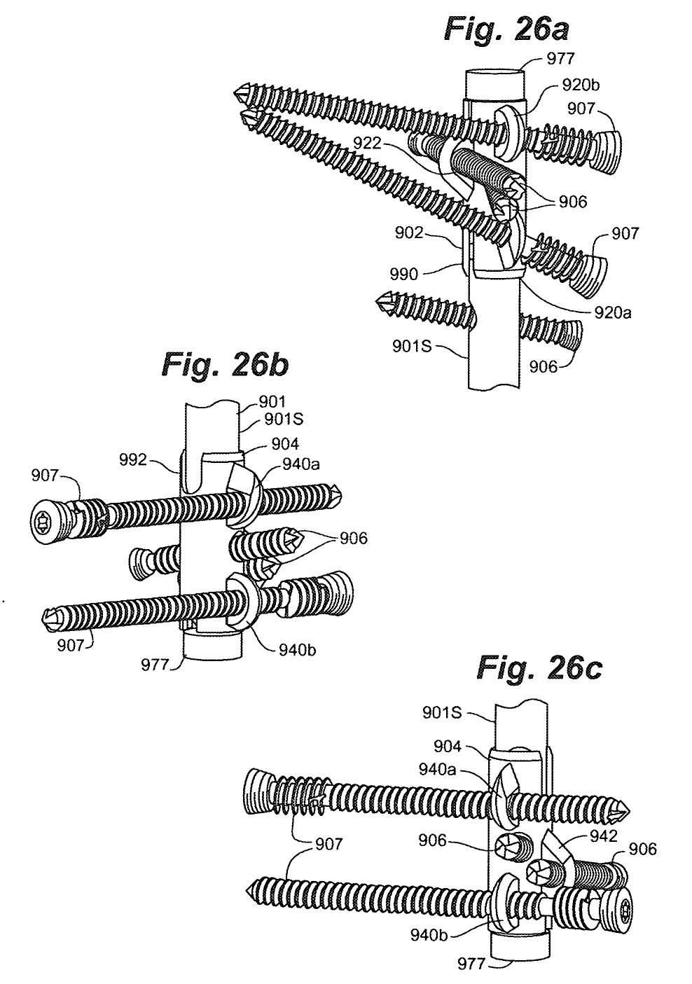

FIGS. 25a-25c are side and end views of another intramedullary nail assembly 900 in accordance with certain embodiments of the present invention. As is shown, the nail assembly 900 is particularly suited for total femoral implantation to facilitate healing of a broken femur 910. FIGS. 26a-26c provide detailed views of the ends of the nail assembly 900. The nail assembly 900 generally includes a bone nail 901 and two anchoring sleeves 902, 904 positioned on the proximal and distal ends of the nail 901. The nail also includes an elongated nail shaft 901S extending between these ends.

The nail shaft 901S has at least one, and preferably multiple, transverse screw holes that pass through the nail shaft 901S (e.g., at an angle oblique to the nail's central axis) and are configured to receive a screw 906 for fixing the nail 901 within the proximal and distal femur or femoral shaft. For example, as shown in FIGS. 25a and 25b, there is a first screw hole 950P positioned in the nail shaft proximate the femoral shaft and another screw hole 950P positioned proximate the lesser trochanter. Turning to FIGS. 26a-26c, the proximal end of the nail shaft (FIG. 26a) includes a transverse screw hole that receives a bone screw 906 through the nail shaft 901S (and also through the sleeve 902) for fixing the proximal end of the nail 901 in the femoral neck. In addition, the distal end of the nail shaft (FIGS. 26b-26c) includes a transverse screw hole that receives a bone screw 906 through the distal end of the nail shaft 901S (and also through the sleeve 902) for fixing the distal end of the nail 901 proximate the intercondylar notch.

In addition, in some cases the nail shaft 901S may also include one or more screw slots (not shown) that pass through the nail shaft 901S as described in more detail with respect to other embodiments herein. Of course, the number and arrangement of screw holes and slots may vary depending upon the particular degree of fixation desired and the scope of the invention is not limited in this regard.

In the illustrated embodiment, a first anchoring sleeve 902 surrounds the proximal end of the nail shaft (which may also be a hollow internally threaded section as described in previous embodiments). The sleeve 902 provides similar functionality to embodiments described elsewhere herein. In particular, the sleeve 902 has three fins 920a, 920b, and 922 that are each provided with an internally threaded mount hole. Each mount hole is configured to receive a desired screw 907 for fixing the proximal end of the nail 901 adjacent to the femoral neck and head. In addition, the second anchoring sleeve 904 surrounds the distal end of the nail shaft (which may also be a hollow internally threaded section as described in previous embodiments). The sleeve 904 provides similar functionality to embodiments described elsewhere herein. In particular, the sleeve 904 has three fins 940a, 940b, and 942 that are each provided with an internally threaded mount hole. Each mount hole is configured to receive a desired screw 907 for fixing the distal end of the nail 901 within the distal supracondylar femur.

As discussed in more detail in previous embodiments, the mount holes are spaced radially from a central axis of the nail shaft 901 (and from a central axis of the sleeves 902, 904). This creates a moment arm between the screws received in the mount holes and the central axis of the nail shaft. The mount holes are preferably internally threaded to provide for positive attachment between the fins and the screws 907 received in the mount holes. Arrangements of this nature provide resistance to micromotion of the nail assembly relative to the bone(s) in which it is fixed. In some cases end caps 977 are provided for closing both the proximal and the distal ends of the nail 901 after implantation.

While embodiments of the present invention has been described, it should be understood that various changes, adaptations and modifications may be made therein without departing from the spirit of the invention and the scope of the appended claims.

What is claimed is:

1. A bone nail assembly comprising:
a bone nail having first and second ends and an elongated nail shaft extending between said ends, the nail shaft having at least one screw hole proximate the second end of the nail shaft and at least one screw hole proximate the first end of the nail shaft, each of said screw holes passing through the nail shaft and being configured to receive a screw for fixing the nail within one or more desired bones, wherein the nail shaft has a hollow internally threaded section adjacent its second end; and
an anchoring sleeve surrounding said hollow internally threaded section of the nail shaft, the anchoring sleeve defining an internal cavity configured to receive a section of the nail shaft, the anchoring sleeve having an outwardly projecting fin, the fin being provided with a mount hole that is internally threaded so as to provide for positive attachment between the fin and a desired screw, said mount hole being configured to receive said desired screw for fixing the nail within said one or more desired bones, the mount hole on the fin being spaced radially from a central axis of the nail shaft thereby creating a moment arm between said desired screw and the central axis of the nail shaft so as to provide resistance to micromotion of the nail assembly relative to said one or more desired bones.

2. The bone nail assembly of claim 1 wherein the mount hole on the fin is spaced at least 5 mm from the central axis of the nail shaft.

3. The bone nail assembly of claim 2 wherein the mount hole on the fin is spaced at least 8 mm from the central axis of the nail shaft.

4. The bone nail assembly of claim 1 wherein the anchoring sleeve has first and second ends, the axial distance between those first and second ends being the sleeve's axial length, said mount hole being located within ¼^{th} of said axial length from the sleeve's second end.

5. The bone nail assembly of claim 1 wherein the anchoring sleeve includes two fins spaced axially apart along a length of the sleeve, such that a first of said two fins is located closer to a first end of the sleeve.

6. The bone nail assembly of claim 5 wherein each of said two fins is provided with a mount hole that is internally threaded, said two mount holes being internally threaded in opposite directions.

7. The bone nail assembly of claim 5 wherein the first of said two fins has a knife edge facing the first end of the sleeve, while the second of said two fins has a generally blunt edge facing the first end of the sleeve.

8. The bone nail assembly of claim 5 wherein a transverse screw opening passes through both the anchoring sleeve and said hollow internally threaded section of the nail shaft, said transverse screw opening being located axially between said two fins.

9. The bone nail assembly of claim 5 wherein each of said two fins has a generally circular or half-circular shape.

10. The bone nail assembly of claim 1 wherein said hollow internally threaded section of the nail shaft is nested concentrically within the anchoring sleeve.

11. The bone nail assembly of claim 1 wherein a transverse screw opening passes through both the anchoring sleeve and said hollow internally threaded section of the nail shaft, the transverse screw opening configured to receive a screw through both the anchoring sleeve and the section of the nail shaft to fix axial movement between the anchoring sleeve and the nail shaft.

12. The bone nail assembly of claim 1 wherein a first end region of the anchoring sleeve defines a slot that is open through a first end of the sleeve, and a corresponding slot passes through the nail shaft, such that when the anchoring sleeve is mounted on the nail shaft said two slots are aligned to define a slot-shaped transverse screw passage.

13. The bone nail assembly of claim 12 wherein said slot in the nail shaft is bounded on two confronting sides by threaded internal wall sections of the nail shaft.

14. The bone nail assembly of claim 12 wherein said slots are configured such that when a transverse screw extends through the slot-shaped passage into the one or more desired bones, the anchoring sleeve and the nail shaft have a desired amount of freedom to move axially relative to the transverse screw to provide bone compression.

15. The bone nail assembly of claim 14 wherein an axial internal compression screw is threadingly received in said hollow internally threaded section of the nail shaft, such that the compression screw and said hollow internally threaded section are threadingly engaged, wherein advancement of the internal compression screw through the hollow internally threaded section toward the transverse screw extending through the slot-shaped passage pulls the first end of the nail shaft toward the transverse screw to provide bone compression.

16. The bone nail assembly of claim 1 wherein an axial compression screw is threadingly received in said hollow internally threaded section of the nail shaft, such that the compression screw and said hollow internally threaded section are threadingly engaged.

17. The bone nail assembly of claim 16 wherein the axial compression screw carries a washer member, wherein in response to a relative rotation of the axial compression screw and the nail shaft, the washer member bears against the one or more desired bones proximate the second end of the nail shaft so as to pull the first end of the nail shaft toward the washer member to provide bone compression.

18. The bone nail assembly of claim 17 wherein the axial compression screw and the washer member are separate bodies that can be disassembled from each other.

19. The bone nail assembly of claim 1 wherein the anchoring sleeve includes two fins angularly spaced about a central axis of the sleeve.

20. The bone nail assembly of claim 19, wherein each of the two fins includes an internally threaded mount hole, the two fins and the respective mount holes oriented such that a screw received within one of the mount holes crosses a screw received within another of the mount holes, for fixing the nail within said one or more desired bones.

21. The bone nail assembly of claim 1 wherein the anchoring sleeve includes a plurality of fins, wherein at least two of the plurality of fins are spaced axially apart along a length of the sleeve, and wherein at least two of the plurality of fins are angularly spaced about a central axis of the sleeve.

22. The bone nail assembly of claim 1 wherein the at least one screw hole proximate the second end of the nail shaft is oriented with respect to the mount hole provided in the fin such that a screw received within the at least one screw hole crosses the desired screw received within the mount hole, for fixing the nail within said one or more desired bones.

23. The bone nail assembly of claim 1 wherein the first end of the nail shaft is a proximal end of the nail shaft, the second end of the nail shaft is a distal end of the nail shaft, and wherein the bone nail assembly is adapted for placement within an ankle.

24. The bone nail assembly of claim 1 wherein the first end of the nail shaft is a distal end of the nail shaft, the second end of the nail shaft is a proximal end of the nail shaft, and wherein the bone nail is adapted for placement within a femur or humerus.

25. A bone nail assembly comprising:
a bone nail having proximal and distal ends and an elongated nail shaft extending between said ends, wherein a screw hole passes through the nail shaft and is configured to receive a screw for fixing the nail within one or more desired bones, the screw hole comprising a threaded first portion and a non-threaded second portion, the non-threaded second portion being bounded by a smooth wall defined by the nail shaft and being configured to receive a screw at different approach angles and guide such screw to the threaded first portion, the non-threaded second portion of the screw hole being devoid of an insert occupying any portion of said screw hole.

26. The bone nail assembly of claim 25 wherein a plurality of screw holes pass through the nail shaft and are each configured to receive a screw for fixing the nail within one or more desired bones, each of said screw holes comprising a threaded first portion and a non-threaded second portion, each non-threaded second portion being bounded by a smooth wall defined by the nail shaft and being configured to receive a screw at different approach angles and guide such screw to the adjacent threaded first portion, each non-threaded second portion being devoid of an insert occupying any portion of the screw hole.

27. The bone nail assembly of claim 26 wherein at least one of said screw holes passes through a hollow internally threaded section of the nail shaft.

28. The bone nail assembly of claim 27 wherein said hollow internally threaded section of the nail shaft is adjacent the distal end of the nail.

29. The bone nail assembly of claim 26 wherein the wall of the non-threaded second portion of at least one of said screw holes is angled inward toward a center of the screw hole from an exterior surface of the nail shaft to the threaded first portion.

30. A bone nail assembly comprising:
a bone nail having proximal and distal ends and an elongated nail shaft extending between said ends, wherein a screw hole passes through the nail shaft and is configured to receive a screw for fixing the nail within one or more desired bones, the screw hole comprising a substantially straight first portion and an angled second portion, the angled second portion being bounded by a wall defined by the nail shaft, the wall being angled inward toward a center of the screw hole from an exterior surface of the nail shaft to the first portion of the screw hole, the angled second portion being configured to receive a screw at different approach angles and guide such screw to the first portion, the angled second portion of the screw hole being devoid of an insert occupying any portion of said screw hole.

31. The bone nail assembly of claim 30, wherein the substantially straight first portion is threaded and the angled second portion is not threaded.

32. The bone nail assembly of claim 30, wherein the substantially straight first portion is threaded and the angled second portion is threaded.

33. A bone nail assembly comprising:
a bone nail having first and second ends and an elongated nail shaft extending between said ends, wherein a screw hole passes through the nail shaft and is configured to receive a screw for fixing the nail within one or more desired bones, the screw hole comprising a threaded first portion and a non-threaded second portion, the non-threaded second portion being bounded by a substantially smooth wall defined by the nail shaft and being configured to receive a screw and guide the screw to the threaded first portion, the non-threaded second portion of the screw hole being devoid of an insert occupying any portion of said screw hole, wherein the nail shaft has a hollow internally threaded section adjacent its second end; and
an anchoring sleeve surrounding said hollow internally threaded section of the nail shaft, the anchoring sleeve defining an internal cavity configured to receive a section of the nail shaft, the anchoring sleeve having an outwardly projecting fin, the fin being provided with a mount hole that is internally threaded so as to provide for positive attachment between the fin and a desired screw, said mount hole being configured to receive said desired screw for fixing the nail within said one or more desired bones, the mount hole on the fin being spaced radially from a central axis of the nail shaft thereby creating a moment arm between said desired screw and the central axis of the nail shaft so as to provide resistance to micromotion of the nail assembly relative to said one or more desired bones.

34. A bone nail assembly comprising:
a bone nail having first and second ends and an elongated nail shaft extending between said ends, the nail shaft having at least one screw hole proximate the second end of the nail shaft and at least one screw hole proximate the first end of the nail shaft, each of said screw holes passing through the nail shaft and being configured to receive a screw for fixing the nail within one or more desired bones; and
an anchoring sleeve defining an internal cavity configured to receive a section of the nail shaft, the anchoring sleeve and the section of the nail shaft each having a transverse screw opening configured to receive a screw through both the anchoring sleeve and the section of the nail shaft to fix axial movement between the anchoring sleeve and the nail shaft, the anchoring sleeve having an outwardly projecting fin, the fin being provided with a mount hole that is internally threaded so as to provide for positive attachment between the fin and a desired screw, said mount hole being configured to receive said desired screw for fixing the nail within said one or more desired bones, the mount hole on the fin being spaced radially from a central axis of the nail shaft thereby creating a moment arm between said desired screw and the central axis of the nail shaft so as to provide resistance to micromotion of the nail assembly relative to said one or more desired bones.
